(19) United States
(12) Patent Application Publication
(10) Pub. No.: US 2017/0326040 A1
(43) Pub. Date: Nov. 16, 2017

(54) PICKERING-TYPE EMULSION COMPRISING A SYNTHETIC PHYLLOSILICATE

(71) Applicant: L'OREAL, Paris (FR)

(72) Inventors: Fabrice SPRINGINSFELD, Chevilly-Larue (FR); Laurence ARNAUD-SEBILLOTTE, Chevilly-Larue (FR); Virginie LECHAUX-TARBOURIECH, Chevilly-Larue (FR)

(73) Assignee: L'OREAL, Paris (FR)

(21) Appl. No.: 15/529,401

(22) PCT Filed: Nov. 24, 2015
(86) PCT No.: PCT/EP2015/077525

§ 371 Date: May 24, 2017

(30) Foreign Application Priority Data
Nov. 24, 2014 (FR) 1461341

(51) Int. Cl. A61K 8/06; A61K 8/26; A61K 8/25; A61Q 19/00; A61Q 15/00

(52) U.S. Cl. CPC A61K 8/06; A61Q 19/00; A61Q 15/00; A61K 8/25; A61K 8/26; A61K 2800/10

(57) ABSTRACT

The invention relates to a Pickering-type emulsion, in particular a cosmetic emulsion, comprising: (a) at least one synthetic phyllosilicate of molecular formula Mg3Si4O10(OH)2; (b) at least one aqueous phase; and (c) at least one oil phase. The invention also relates to the use of the synthetic phyllosilicate as an agent for stabilising the emulsion, a method for producing the emulsion, and a cosmetic treatment method comprising the application of the emulsion to the skin and/or nails.

The present invention relates to compositions, in particular cosmetic or dermatological compositions, preferably for topical application, of the “Pickering” type. More specifically, the present invention relates to the area of caring for, cleansing, protecting and/or making up the skin and/or nails, and particularly the skin of the face and/or body.

The term “skin” means the skin of the face and/or of the body.

The term “nails” also means false nails in so far as the cosmetic effects desired are often the same.

In the cosmetic field, it is conventional to formulate oil-in-water (O/W) or water-in-oil (W/O) emulsions. These emulsions are more particularly interesting for their sensory properties. However, these cosmetic emulsions are, generally, stabilized by surfactants that can be a drag on some applications.

Indeed surfactants, specifically as emulsifiers may affect the cosmeticity of products and the powdery nature of deposit, greatly favored by consumers. Specifically, in the case of antiperspirants, the presence of aluminum salts limits the use of a broad family of surfactants because of their ionic nature. These surfactants may also cause some harmful effects, such as a sticky, slippery or soapy effect.

Therefore, there is a need for stable emulsions and compositions that respect the skin and/or nails and in doing so have reduced quantities of surfactants or are advantageously free of surfactants.

Accordingly, using particles at the interfaces of these emulsions has been proposed, to make Pickering emulsions. A Pickering emulsion is an emulsion comprising a continuous phase and a dispersed phase in said continuous phase; said phases are not miscible, and said emulsion is stabilized by solid particles adsorbed at the interface of the two non-miscible phases and without it being necessary to use emulsifying surfactants. In addition to this contribution to the stability at interfaces, the particles considered in Pickering emulsions also participate in the sensory aspect of formulas as cosmetic fillers.

Application EP1005849 in particular teaches the use of compositions comprising Pickering emulsions for treating the hair and scalp.

Nevertheless, few amphiphilic mineral particles exist that meet non-nanometric cosmetic criteria and remain small enough to produce homogeneous, cosmetic and stable creams/emulsions.

In a surprising manner, the inventors have recently observed that using a synthetic phyllosilicate with molecular formula Mg₃Si₄O₁₀(OH)₂ in a Pickering emulsion improves the stability of said emulsions.

Accordingly the present invention relates to a Pickering emulsion, in particular a cosmetic emulsion, comprising:
(a) at least one synthetic phyllosilicate having molecular formula Mg₃Si₄O₁₀(OH)₂,
(b) at least one aqueous phase, and
(c) at least one oil phase,

Advantageously, the composition according to the present invention comprising said phyllosilicate presents an X-ray diffraction line greater than 9.4 Å and less than or equal to 9.8 Å.

Advantageously, the composition comprising the emulsion according to the present invention comprising said phyllosilicate has an infrared absorption band at 7200 cm⁻¹, corresponding to the stretching vibration attributed to the silanol groups Si—OH at the edge of the sheets.

Advantageously, the composition comprising the emulsion according to the present invention comprising said phyllosilicate is characterized by the absence of an infrared absorption band at 7156 cm^{31 1}. This band at 7156 cm⁻¹ corresponds to the vibration band of Mg₂FeOH.

The composition comprising the emulsion according to the present invention comprising said phyllosilicate also preferably has an infrared absorption band at 7184 cm⁻¹ corresponding to the 2v Mg₃OH stretching vibration.

It should be noted that in the presence of adsorbed water, for example residual water, a broad infrared absorption band is detectable and readily identifiable, for example at 5500 cm⁻¹.

According to the present invention, particles of synthetic phyllosilicate are adsorbed at the interface of the aqueous phase and the oil phase which stabilizes said emulsion and produces a macroscopically homogeneous composition.

According to a preferred embodiment of the invention, the Pickering emulsion does not contain any emulsifying surfactant.

“Does not contain any emulsifying surfactant” means containing less than 1.00% by weight, preferably less than 0.50% by weight and even more preferably less than 0.10% by weight of emulsifying surfactant or even being totally free of emulsifying surfactant.

For the purposes of the present invention, the term “surfactant” means an amphiphilic molecule, i.e. a molecule that has two parts of different polarity, one being lipophilic (which retains fatty substances) and non-polar, and the other hydrophilic (water-miscible) and polar.

Surfactants are characterized by their HLB (hydrophilic-lipophilic balance) value, the HLB being the ratio between the hydrophilic part and the lipophilic part in the molecule. The term “HLB” is well known to those skilled in the art and is described, for example, in “The HLB system. A time-saving guide to Emulsifier Selection” (published by ICI Americas Inc.; 1984). For emulsifiers, the HLB generally ranges from 3 to 8 for the preparation of W/O emulsions and from 8 to 18 for the preparation of O/W emulsions. The HLB of the surfactant(s) used according to the invention may be determined via the Griffin method or the Davies method.

The presence of a synthetic phyllosilicate having molecular formula Mg₃Si₄O₁₀(OH)₂ is therefore particularly advantageous to improve the stability of Pickering emulsions, and the stability of compositions comprising said emulsions.

In an equally surprising manner, it has been observed that the compositions comprising these emulsions do not have any sticky effect, and do not “slip.”

A synthetic phyllosilicate suitable for the invention may be used in a composition comprising the emulsion according to the invention in the form of powder or in the form of aqueous or aqueous-alcoholic gel and advantageously in the form of aqueous or aqueous-alcoholic gel.

It has also been shown that said synthetic phyllosilicate in gel form was particularly effective for stabilizing said oil phase/aqueous phase interfaces, and to form fine emulsions that are stable over time.

Therefore the use of these synthetic phyllosilicates reduces the need for surfactants, particularly ionic and/or nonionic surfactants, and therefore improves ease of use of compositions comprising the emulsion according to the invention.

Synthetic phyllosilicates such as those described in patent application WO 2008/009799 and advantageously those disclosed in patent application FR 2 977 580 are most particularly suitable for use in the invention.

However, neither of these documents WO2008/009799 and FR 2 977 580 considers exploiting these resulting synthetic phyllosilicates in compositions relating to cosmetic, dermatological or pharmaceutical applications.

Specifically, none of these documents considers using this synthetic phyllosilicate in a Pickering emulsion.

According to a first variant, said synthetic phyllosilicate is used there in the form of an aqueous or aqueous-alcoholic gel.

According to a second variant, said synthetic phyllosilicate is used there in a dry (or powder) particulate form.

According to a third variant, said synthetic phyllosilicate is used there in the form of an aqueous or aqueous-alcoholic gel and in a dry (or powder) particulate form.

According to an embodiment, the composition comprising the emulsion according to the invention is a cosmetic or dermatological composition comprising a physiologically acceptable medium.

The present invention also relates to the use of a synthetic phyllosilicate having molecular formula Mg₃Si₄O₁₀(OH)₂ in accordance with the invention in a Pickering emulsion, as a stabilizer for said emulsion.

According to another feature, the invention relates to a process for preparing an emulsion according to the invention, comprising the following steps:

1) In water, with stirring, the synthetic phyllosilicate having a molecular formula Mg₃Si₄O₁₀(OH)₂ in accordance with the invention as defined below and optionally an additional gellant are dispersed with stirring in a Rayneri;

2) The aqueous phase is mixed with the oil phase;

3) The emulsion is formed;

(i) at a temperature of 20-25° C., if there is no fatty substance that is solid, with stirring;

(ii) at a higher temperature than the melting point of said fatty substance.

According to another feature, the invention relates to a process for cosmetic treatment comprising the application of an emulsion according to the invention to the skin and/or nails.

Synthetic Phyllosilicate

The synthetic phyllosilicate in accordance with the invention has a crystalline structure in accordance with that of a hydroxylated magnesium silicate of molecular formula Mg₃Si₄O₁₀(OH)₂ belonging to the chemical family of phyllosilicates.

These phyllosilicates are generally formed from a stack of elemental leaflets of crystalline structure, the number of which ranges from a few units to several tens of units. Each elemental leaflet is formed by the association of two layers of tetrahedra in which the silicon atoms are positioned, located on either side of a layer of octahedra in which the magnesium atoms are positioned. This group corresponds to the 2/1 phyllosilicates, which are also termed as being of T.O.T. type. (tetrahedron-octahedron-tetrahedron).

As presented above, a synthetic phyllosilicate in accordance with the invention may be obtained according to a preparation process such as the one described in patent application WO 2008/009799 and is preferentially obtained according to the technology described in patent application FR 2 977 580.

This preparation process especially comprises a prolonged hydrothermal treatment, which makes it possible to obtain an aqueous gel of synthetic phyllosilicate. Accordingly, according to a first embodiment, the synthetic phyllosilicate may be used in the form of an aqueous or aqueous-alcoholic gel, especially like the one obtained directly on conclusion of the synthetic process.

As described in patent application FR 2 977 580, the parameters that influence the synthesis and the properties of a synthetic phyllosilicate in gel form that is suitable for use in the invention are the nature of the heat treatment (200° C. to 900° C.), the pressure, the nature of the reagents and the proportions thereof.

More particularly, the duration and temperature of the hydrothermal treatment make it possible to control the size of the particles. For example, the lower the temperature, the smaller the synthesized particles, as described in patent application FR 2 977 580. Controlling the size makes it possible to afford new properties and allows for better control of both its hydrophilic and hydrophobic properties, i.e. amphiphilic properties.

It should nevertheless be noted that the gel as obtained after the synthesis process may be subjected to an optional washing step with water/centrifugation, after which it is dried and milled. The synthetic phyllosilicate is then available in the form of powder.

Accordingly, the synthetic phyllosilicate considered according to the invention may also be formulated in the state of powder within a composition according to the invention.

Structural Analysis and Characterization of a Synthetic Phyllosilicate that is Suitable for Use in the Invention

A synthetic phyllosilicate that is suitable for use in the invention may be characterized by various parameters, namely infrared absorption bands, its size and its purity, as detailed below.

Under certain conditions, analyses such as nuclear magnetic resonance in particular of ²⁹Si may be useful for the characterization of a synthetic phyllosilicate that is suitable for use in the invention. Similarly, thermogravimetric analysis (TGA) may be used for the characterization of a synthetic phyllosilicate that is suitable for use in the invention. Finally, x-ray diffraction may also be used for this purpose.

Infrared
Method Used

The machine used is a Nicolet 6700 FTIR Fourier transform spectrometer, equipped with an integration sphere, with an InGaA detector and a CaF₂ separator and a resolution of 12 cm⁻¹, more preferentially of 8 cm⁻¹ and even more preferentially of 4 cm⁻¹. In other words, the values of the absorption bands given in this description should be considered as being approximately 6 cm⁻¹ and more preferentially approximately 4 cm⁻¹ and even more preferentially approximately 2 cm⁻¹.

The near infrared recordings of the stretching region located at 7184 cm⁻¹ were broken down by pseudo-Voigt functions using the Fityk software (Wojdyr, 2010).

To visualize the absorption spectrum in a composition comprising at least one aqueous part, such as an emulsion, it is recommended to heat this composition to a temperature corresponding to a temperature of greater than or equal to 100° C. (for example 120° C.) and less than or equal to 500° C. (for example 400° C.) so as to remove the adsorbed water part and, where appropriate, some or all of the organic compound(s) present in the composition.

Generally to confirm an infrared absorption band, the person skilled in the art performs stretching enlargements, specifically, he may for example enlarge by approximately 200 cm⁻¹ either side of the suspected infrared absorption band. A natural talc is a mineral species composed of doubly hydroxylated magnesium silicate having formula Mg₃Si₄O₁₀(OH)₂, which may contain traces of nickel, iron, aluminum, calcium or sodium.

Natural talc has an infrared spectrum with a typical, fine and strong absorption band at 7184 cm⁻¹ corresponding to the 2v Mg₃OH stretching vibration. Natural talc also contains chemical elements which replace magnesium and silicon in the crystalline structure, which impose the appearance of at least one additional infrared absorption band, in particular that corresponding to the stretching vibration at 7156 cm⁻¹ attributable to 2v Mg₂FeOH.

The spectrum of the synthetic phyllosilicate that is suitable for use in the invention differs from that of a natural talc by an infrared absorption band at 7200 cm⁻¹ corresponding to the stretching vibration attributed to the silanol groups Si—OH at the edge of the phyllosilicate leaflets.

To confirm this infrared absorption band, a person skilled in the art may perform a stretching amplification, in particular in the region 7400 cm⁻¹-7000 cm⁻¹ and more particularly in the region 7300 cm⁻¹-7100 cm⁻¹.

Preferably, the spectrum of the synthetic phyllosilicate is also characterized by an absence of infrared absorption band at 7156 cm⁻¹. This band at 7156 cm⁻¹ corresponds to the vibration band of Mg₂FeOH.

Preferably, the spectrum of the synthetic phyllosilicate is also characterized by the absorption band at 7184 cm⁻¹ which is common to natural talc.

It should be noted that in the presence of adsorbed water, for example residual water, a broad infrared absorption band is detectable and readily identifiable, for example at 5500 cm⁻¹.

Advantageously, the composition according to the present invention comprising said phyllosilicate has an infrared absorption band at 7200 cm⁻¹, corresponding to the stretching vibration attributed to the silanol groups Si—OH at the edge of the phyllosilicate sheets.

Advantageously, the composition according to the present invention comprising said phyllosilicate is characterized by the absence of an infrared absorption band at 7156 cm⁻¹. This band at 7156 cm⁻¹ corresponds to the vibration band of Mg₂FeOH.

The composition according to the present invention comprising said phyllosilicate also preferably has an infrared absorption band at 7184 cm⁻¹ corresponding to the 2v Mg₃OH stretching vibration.

In a composition according to the invention, it should be noted that in the presence of adsorbed water, for example residual water, a broad infrared absorption band is detectable and readily identifiable, for example at 5500 cm⁻¹.

Size

Method Used

To perform the particle size analysis of the synthetic phyllosilicates that are suitable for use in the invention, photon correlation spectroscopy was used. This analytical technique affords access to the size of the particles on the basis of the principle of dynamic light scattering. This device measures over time the intensity of the light scattered by the particles at an angle θ under consideration and the scattered rays are then processed using the Padé-Laplace algorithm.

This non-destructive technique requires dissolution of the particles. The particle size measurement obtained by this technique corresponds to the value of the hydrodynamic diameter of the particle, i.e. it comprises both the particle size and also the thickness of the hydration layer.

The analyses were performed using a VASCO-2 particle size analyzer from Cordouan. For the purpose of obtaining statistical information regarding the particle distribution, the NanoQ™ software was used in multi-acquisition mode with the Padé-Laplace algorithm.

Thus, a synthetic phyllosilicate that is suitable for use in the invention, in the form of an aqueous or aqueous-alcoholic gel, advantageously has a mean size ranging from 300 nm to 500 nm.

By contrast, a synthetic phyllosilicate when it is used in the form of a powder, in the image of that obtained by dehydration of an aqueous gel, as defined above, may have an average size ranging from a few microns to several hundred microns, preferably ranging from 5 μm to 100 μm, or may be presented in the form of porous micron or multimicron aggregates composed of said particles.

These characteristics are advantageous with regard to a natural talc, one of the constraints of which is the uncontrolled size of its particles.

Purity

The synthetic phyllosilicate under consideration according to the invention has a degree of purity of at least 99.90% and preferably of at least 99.99%.

It is thus advantageously free of impurities or of undesirable compounds, among which are especially asbestos minerals such as asbestos (serpentine), chlorite, carbonates, heavy metals, iron sulfides, etc., which are generally associated with natural talc and/or incorporated into the structure of natural talcs.

NMR (Nuclear Magnetic Resonance)

Methods Used

The silicon-29 (²⁹Si) NMR spectra were recorded on a Bruker Avance 400 (9.4 T) spectrometer. The reference for the chemical shifts is tetramethylsilane (TMS). The samples were placed in 4 mm zirconia rotors. The magic angle spinning (MAS) speed was set at 8 kHz. The experiments were performed at a room temperature of 21° C.

The ²⁹Si spectra were obtained either by direct polarization (rotation of 30°) with a recycling delay of 60 seconds, or by cross polarization (CP) between 1H and ²⁹Si (recycling time of 5 seconds and contact time of 3 ms).

In silicon (²⁹Si) NMR, natural talc has a single peak at −97 ppm.

In silicon (²⁹Si) NMR, in contrast with natural talc, the spectrum of the synthetic phyllosilicate in accordance with the invention shows two peaks: one located at −95 ppm and the other located at −97 ppm, this being the case without the need for particle size fractionation to a size of less than 500 nm.

TGA (Thermogravimetric Analysis)

Method Used

The recordings were made using a Perkin Elmer Diamonds thermobalance. For each analysis, about 20 mg of sample were required. During the analysis, the sample is subjected to a temperature increase ranging from 30° C. to 1200° C. at a rate of 10° C.min⁻¹ under a stream of 100 mL.min⁻¹ of air.

The thermogravimetric analysis of a synthetic phyllosilicate in accordance with the invention shows lower thermal stability (at about 800° C.) than that of natural talc and it is characterized by four losses of mass, in contrast with natural talc which has only one, at about 900° C.

To establish these losses of mass, it is useful to refer to the article by Angela Dumas, Francois Martin, Christophe Le Roux, Pierre Micoud, Sabine Petit, Eric Ferrage, Jocelyne Brendle, Olivier Grauby and Mike Greenhill-Hooper: “Phyllosilicates synthesis: a way of accessing edges contributions in NMR and FTIR spectroscopies. Example of synthetic talc” (Phys. Chem. Minerals, published on 27 Feb. 2013).

X-ray Diffraction

Method Used

Analysis of the x-ray diffractogram, especially with the aid of the materials and method used for x-ray diffraction analysis, is detailed in patent application FR 2 977 580.

Preferably, given that X-ray diffractogram analysis is performed only on solids, to visualize the absorption spectrum in a composition comprising at least one aqueous part, such as an emulsion, it is recommended to heat this composition to a temperature corresponding to a temperature greater than or equal to 100° C. (for example 120° C.) and less than or equal to 500° C. (for example 400° C.) to remove the absorbed water part and, where appropriate, some or all of the organic compound(s) present in the composition.

The x-ray diffractogram of the synthetic phyllosilicate that is suitable for use in the invention has the same positions of the diffraction lines as those of natural talc, with the exception of one line. Specifically, natural talc has a diffraction line at 9.36 Å whereas the synthetic phyllosilicate in accordance with the invention has a diffraction line above 9.4 Å, which may be up to 9.8 Å.

More particularly, the synthetic phyllosilicate in accordance with the invention has a diffraction line greater than 9.4 Å and less than or equal to 9.8 Å.

The synthetic phyllosilicate in accordance with the invention preferably has a diffraction line greater than or equal to 9.5 Å, advantageously greater than or equal to 9.6 Å, and preferentially greater than or equal to 9.7 Å.

The synthetic phyllosilicate in accordance with the invention preferably has a diffraction line less than or equal to 9.7 Å, advantageously less than or equal to 9.6 Å, and preferentially less than or equal to 9.5 Å.

The synthetic phyllosilicate in accordance with the invention may also have a diffraction line between 4.60 Å and 4.80 Å, and/or a diffraction line between 3.10 Å and 3.20 Å and/or a diffraction line between 1.51 Å and 1.53 Å.

It should be noted that a synthetic phyllosilicate in accordance with the invention is free of interfoliar cations. Specifically, this characteristic is demonstrated by the absence of an x-ray diffraction line located at a distance of between 12.00 Å and 18.00 Å, usually revealing a swelling phase with interfoliar spaces in which are found interfoliar cations and possible water molecules.

It is understood that when a synthetic phyllosilicate in accordance with the invention is in gel form, the “weight %” means the “weight % of solids” or “weight % of active ingredient”.

When a synthetic phyllosilicate suitable for use in the invention is in aqueous or aqueous-alcoholic gel form, it is present in a quantity ranging from 0.5% to 10% by weight of active ingredient, more preferably from 1% to 7.5% by weight of active ingredient, and better from 2% to 4% by weight of active ingredient relative to the total weight of the composition for 0.3% to 50% by weight of oil phase, preferentially for 2% to 25% by weight of oil phase and better still for 4% to 20% by weight of oil phase.

When a synthetic phyllosilicate suitable for use in the invention is in anhydrous form (powder), it is present in a quantity ranging from 0.1% to 40% by weight of active ingredient, in particular from 0.5% to 35% by weight of active ingredient, preferably from 1% to 32% by weight of active ingredient and more preferentially from 2% to 30% by weight of active ingredient, relative to the total weight of the composition.

Physiologically Acceptable Medium

As presented above, a composition according to the invention may advantageously be a cosmetic or dermatological composition.

In this particular embodiment, since a composition according to the invention is intended for topical application to the skin and/or the nails, it contains a physiologically acceptable medium.

For the purposes of the present invention, the term “physiologically acceptable medium” means a medium that is compatible with the skin and/or the nails.

Thus, the physiologically acceptable medium is in particular a cosmetically or dermatologically acceptable medium, i.e. a medium that has no unpleasant odor, color or appearance, and that does not cause the user any unacceptable stinging, tautness or redness.

Aqueous Phase

The aqueous phase of a composition according to the invention comprises water and optionally a water-soluble solvent.

In the present invention, the term “water-soluble solvent” denotes a compound that is liquid at room temperature and water-miscible (miscibility with water of greater than 50% by weight at 25° C. and atmospheric pressure).

The water-soluble solvents that may be used in the composition of the invention may also be volatile.

Among the water-soluble solvents that may be used in the composition in accordance with the invention, mention may be made especially of lower monoalcohols containing from 1 to 5 carbon atoms such as ethanol and isopropanol, glycols containing from 2 to 8 carbon atoms such as ethylene glycol, propylene glycol, 1,3-butylene glycol and dipropylene glycol, C₃ and C₄ ketones and C₂-C₄ aldehydes.

The aqueous phase (water and optionally the water-miscible solvent) may be present in the composition in a content ranging from 5% to 95%, better still from 30% to 80% by weight and preferably from 40% to 75% by weight relative to the total weight of said composition.

According to another embodiment variant, the aqueous phase of a composition according to the invention may comprise at least one C₂-C₃₂ polyol.

For the purposes of the present invention, the term “polyol” should be understood as meaning any organic molecule comprising at least two free hydroxyl groups.

Preferably, a polyol in accordance with the present invention is present in liquid form at room temperature.

A polyol that is suitable for use in the invention may be a compound of linear, branched or cyclic, saturated or unsaturated alkyl type, bearing on the alkyl chain at least two —OH functions, in particular at least three —OH functions and more particularly at least four —OH functions.

The polyols that are advantageously suitable for formulating a composition according to the present invention are those especially containing from 2 to 32 carbon atoms and preferably 3 to 16 carbon atoms.

Advantageously, the polyol may be chosen, for example, from ethylene glycol, pentaerythritol, trimethylolpropane, propylene glycol, 1,3-propanediol, butylene glycol, isoprene glycol, pentylene glycol, hexylene glycol, glycerol, polyglycerols such as glycerol oligomers, for instance diglycerol, and polyethylene glycols, and mixtures thereof.

According to a preferred embodiment of the invention, said polyol is chosen from ethylene glycol, pentaerythritol, trimethylolpropane, propylene glycol, glycerol, polyglycerols, polyethylene glycols and mixtures thereof.

According to a particular mode, the composition of the invention may comprise at least propylene glycol.

According to another particular mode, the composition of the invention may comprise at least glycerol.

According to a specific embodiment, a synthetic phyllosilicate suitable for use in the invention is the form of an aqueous or aqueous-alcoholic gel, and constitutes all or part of the aqueous phase.

According to a specific embodiment, a synthetic phyllosilicate that is suitable for use in the invention in the form of an aqueous or aqueous-alcoholic gel, constitutes all or part of the aqueous phase, i.e. the aqueous phase of the emulsion is exclusively constituted of this gel.

Fatty Phase

For the purposes of the invention, a fatty phase includes any liquid fatty substance, generally oils (also known as liquid or oily fatty phase), or solid fatty substance like waxes or pasty compounds (also known as solid fatty phase).

In the sense of the invention, a liquid fatty phase is also called oil phase and comprises at least one oil.

The term “oil” means any fatty substance that is in liquid form at room temperature and atmospheric pressure.

An oily phase that is suitable for preparing the cosmetic compositions according to the invention may comprise hydrocarbon-based oils, silicone oils, fluoro oils or non-fluoro oils, or mixtures thereof

The oils may be volatile or non-volatile.

They may be of animal, plant, mineral or synthetic origin. According to one implementation variant, oils of plant origin are preferred.

For the purposes of the present invention, the term “nonvolatile oil” means an oil with a vapor pressure of less than 0.13 Pa.

For the purposes of the present invention, the term “silicone oil” means an oil comprising at least one silicon atom, and in particular at least one Si—O group.

The term “fluoro oil” means an oil comprising at least one fluorine atom.

The term “hydrocarbon-based oil” means an oil mainly containing hydrogen and carbon atoms.

The oils may optionally comprise oxygen, nitrogen, sulfur and/or phosphorus atoms, for example in the form of hydroxyl or acid radicals.

For the purposes of the invention, the term “volatile oil” means any oil that is capable of evaporating on contact with the skin in less than one hour, at room temperature and atmospheric pressure. The volatile oil is a volatile cosmetic compound, which is liquid at room temperature, especially having a nonzero vapor pressure, at room temperature and atmospheric pressure, especially having a vapor pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

Volatile Oils

The volatile oils may be hydrocarbon-based oils or silicone oils.

Among the volatile hydrocarbon-based oils containing from 8 to 16 carbon atoms, mention may be made especially of branched C₈-C₁₆ alkanes, for instance C₈-C₁₆ isoalkanes (also known as isoparaffins), isododecane, isodecane, isohexadecane and, for example, the oils sold under the trade names Isopar or Permethyl, branched C₈-C₁₆ esters, for instance isohexyl neopentanoate, and mixtures thereof. Preferably, the volatile hydrocarbon-based oil is selected from volatile hydrocarbon-based oils containing from 8 to 16 carbon atoms, and mixtures thereof, in particular from isododecane, isodecane and isohexadecane, and is especially isohexadecane.

Mention may also be made of volatile linear alkanes comprising from 8 to 16 carbon atoms, in particular from 10 to 15 carbon atoms and more particularly from 11 to 13 carbon atoms, for instance n-dodecane (C₁₂) and n-tetradecane (C₁₄) sold by Sasol under the respective references Parafol 12-97 and Parafol 14-97, and also mixtures thereof, the undecane-tridecane mixture, mixtures of n-undecane (C₁₁) and of n-tridecane (C₁₃) obtained in Examples 1 and 2 of patent application WO 2008/155 059 from the company Cognis, and mixtures thereof.

Volatile silicone oils that may be mentioned include linear volatile silicone oils such as hexamethyldisiloxane, octamethyltrisiloxane, decamethyltetrasiloxane, tetradecamethylhexasiloxane, hexadecamethylheptasiloxane and dodecamethylpentasiloxane.

Volatile cyclic silicone oils that may be mentioned include hexamethylcyclotrisiloxane, octamethylcyclotetrasiloxane, decamethylcyclopentasiloxane and dodecamethylcyclohexasiloxane.

Nonvolatile Oils

The non-volatile oils may, in particular, be chosen from non-volatile hydrocarbon-based, fluoro and/or silicone oils.

Non-volatile hydrocarbon-based oils that may especially be mentioned include:
hydrocarbon-based oils of animal origin,
hydrocarbon-based oils of plant origin, synthetic ethers containing from 10 to 40 carbon atoms, such as dicaprylyl ether,
synthetic esters, such as the oils having formula R₁COOR₂, in which R₁ represents a linear or branched fatty acid residue comprising from 1 to 40 carbon atoms and R₂ represents a hydrocarbon-based chain, which is in particular branched, containing from 1 to 40 carbon atoms, on condition that R₁+R₂ is ≧10. The esters may be chosen especially from fatty acid and fatty alcohol esters, such as for example cetostearyl octanoate, isopropyl alcohol esters such as isopropyl myristate or isopropyl palmitate, ethyl palmitate, 2-ethylhexyl palmitate, isopropyl stearate, octyl stearate, hydroxylated esters, such as isostearyl lactate or octyl hydroxystearate, alkyl or polyalkyl ricinoleates, hexyl laurate, neopentanoic acid esters, such as isodecyl neopentanoate or isotridecyl neopentanoate, and isononanoic acid esters, such as isononyl isononanoate or isotridecyl isononanoate,
polyol esters and pentaerythritol esters, such as dipentaerythrityl tetrahydroxystearate/tetraisostearate,
non-phenyl silicone oils, for instance caprylyl methicone, and
phenyl silicone oils, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, dimethicones or phenyl trimethicone with a viscosity of less than or equal to 100 cSt, and trimethyl-pentaphenyl-trisiloxane, and mixtures thereof; and also mixtures of these various oils.

Preferably, a composition according to the invention comprises volatile and/or non-volatile silicone oils.

A composition according to the invention may comprise from 5% to 95% by weight, better still from 5% to 40% by weight, and preferably from 7% to 35% by weight of oil(s) relative to the total weight of said composition.

Waxes

For the purposes of the present invention, the term “wax” means a lipophilic fatty compound that is solid at room temperature (25° C.), with a reversible solid/liquid change of state, having a melting point of greater than 30° C. which may be up to 200° C., a hardness of greater than 0.5 MPa, and having an anisotropic crystal organization in the solid state. By bringing the wax to its melting point, it is possible to make it miscible with oils and to form a microscopically homogeneous mixture, but on returning the temperature of the mixture to room temperature, recrystallization of the wax in the oils of the mixture is obtained.

The waxes that may be used in the invention are compounds that are solid at room temperature, which are intended to structure the composition, in particular in stick form; they may be hydrocarbon-based, fluoro- and/or silicone-based and may be of plant, mineral, animal and/or synthetic origin. In particular, they have a melting point of greater than 40° C. and better still greater than 45° C.

As wax that may be used in the invention, mention may be made of those generally used in cosmetics: they are especially of natural origin, such as beeswax, carnauba wax, candelilla wax, ouricury wax, Japan wax, cork fiber wax or sugarcane wax, rice wax, montan wax, paraffin, lignite wax or microcrystalline wax, ceresin or ozokerite, hydrogenated oils such as jojoba oil; synthetic waxes such as polyethylene waxes derived from the polymerization or copolymerization of ethylene and Fischer-Tropsch waxes, or alternatively fatty acid esters such as octacosanyl stearate, glycerides that are solid at 40° C. and better still at 45° C., silicone waxes such as alkyl or alkoxy dimethicones with an alkyl or alkoxy chain of 10 to 45 carbon atoms, poly(di)methylsiloxane esters that are solid at 40° C., the ester chain of which comprises at least 10 carbon atoms; and mixtures thereof.

As a guide, a composition according to the invention may comprise from 0.01% to 50% by weight, preferably from 2% to 40% by weight, and better still from 5% to 30% by weight of wax(es), relative to the total weight of the composition.

Pasty Compound

For the purposes of the present invention, the term “pasty” is intended to denote a lipophilic fatty compound with a reversible solid/liquid change of state, and comprising at a temperature of 23° C. a liquid fraction and a solid fraction.

The pasty compound is advantageously chosen from:
lanolin and derivatives thereof,
polymeric or non-polymeric fluoro compounds,
polymeric or non-polymeric silicone compounds,
vinyl polymers, especially:
olefin homopolymers,
olefin copolymers,
hydrogenated diene homopolymers and copolymers;
linear or branched homopolymer or copolymer oligomers of alkyl (meth)acrylates preferably bearing a C₈-C₃₀ alkyl group,
homopolymer and copolymer oligomers of vinyl esters bearing C₈-C₃₀ alkyl groups,
homopolymer and copolymer oligomers of vinyl ethers bearing C₈-C₃₀ alkyl groups,
liposoluble polyethers resulting from polyetherification between one or more C₂-C₁₀₀ and preferably C₂-C₅₀ diols,
esters,
polyvinyl laurate; and
mixtures thereof.
   As a guide, a composition according to the invention may comprise from 1% to 99% by weight, preferably from 1% to 60% by weight, better from 2 to 30% and better still from 5% to 20% by weight of pasty compounds, relative to the total weight of the composition.

The liquid fatty phase may contain other compounds dissolved in the oils, such as gelling agents and/or structuring agents. These compounds may be chosen especially from gums, such as silicone gums (dimethiconol); silicone resins, such as trifluoromethyl(C1-C4 alkyl) dimethicone and trifluoropropyl dimethicone, and silicone elastomers, for instance the products sold under the KSG names by the company Shin-Etsu, under the name Trefil by the company Dow Corning or under the Gransil names by the company Grant Industries; and mixtures thereof.

These fatty substances mentioned above can be chosen in a varied way by the person skilled in the art so as to prepare a composition having the desired properties, for example of consistency or texture.

For emulsions, the proportion of oil phase will be chosen according to the type of emulsion. According to a preferred embodiment of the invention, the emulsion will be an oil-in-water emulsion.

The oil phase may therefore be present in the composition in a quantity ranging from 1% to 80% and better still from 5% to 70% by weight relative to the total weight of the composition.

Preferably, the oil phase comprises an oil chosen from alkanes, such as isohexadecane, esters such as isopropyl palmitate, ethers such as dicaprylyl ether, triglycerides such as capric/caprylic acid triglyceride and silicones, specifically non-volatile silicones such as polydimethylsiloxanes, such as PDMS 6 cst.

Additives

The compositions according to the invention may, in addition, also comprise additional cosmetic and dermatological active ingredients.

The cosmetic compositions according to the invention may comprise cosmetic adjuvants chosen from opacifiers, stabilizers, preservatives, polymers, fragrances, thickeners, gellants, sunscreens, dermatological or cosmetic active agents, fillers, suspension agents, colorants or any other ingredient usually used in cosmetics for this type of application.

Among gellants, water-soluble polysaccharides will in particular be used.

The term “polysaccharide” means any polymer consisting of several saccharides (or monosaccharides) having the general formula: —[Cₓ(H₂O)_{y})]ₙ- (in which y is generally x−1) and linked together via O-oside bonds.

“Water-soluble” means partially or totally soluble in water to give a solution gelled or thickened at a concentration of 1% of active ingredient in water, after use when cold or hot.

Water-soluble polysaccharides that can be used in the present invention are chosen in particular from starches, gellans, scleroglucan gum, guar gum, konjac, agar agar, celluloses such as hydroxyethylcellulose, hydroxypropylcellulose, and mixtures thereof.

In a preferred manner, unmodified starches, unmodified starches and mixtures thereof.

Accordingly, according to a preferred embodiment, an emulsion according to the invention further comprises at least one water-soluble polysaccharide, specifically and modified or unmodified starch.

Among the unmodified starches, mention may be made of unmodified corn starches (INCI name: Zea mays starch), for instance the products sold under the trade name Farmal CS®, in particular the commercial product Farmal CS 3650® from the company Corn Products International. Mention may also be made of unmodified rice starches (INCI name: Oryza sativa (rice) starch), for instance the commercial product Remy DR I® sold by the company Beneo-Remy.

Among unmodified starches, mention may be made of distarch phosphates or compounds rich in distarch phosphate, in particular the hydroxypropyl ethers of distarch phosphate having the INCI name: Hydroxypropyl Starch Phosphate, for instance the products sold under the trade names Farinex VA70 C or Farmal MS 689® from the company AVEBE Stadex; or the products sold under the trade names Structure BTC®, Structure HVS®, Structure XL® or Structure Zea® from National Starch (corn distarch phosphate).

According to the invention, the water-soluble polysaccharide(s) may represent from 0.5% to 6% by weight and more particularly from 1% to 4% by weight relative to the total weight of the final composition.

Composition

The aqueous compositions comprising the emulsion according to the invention may be prepared according to the techniques that are well known to those skilled in the art.

For example, when the composition comprising the emulsion according to the invention is cosmetic or dermatological, it may be in the form of an oil-in-water or a water-in-oil emulsion with liquid to semi-solid consistency.

Preferably, the composition is in the form of an oil-in-water (direct emulsion (O/W)) or water-in-oil (inverse emulsion (W/O)) emulsion and more preferably oil-in-water.

The cosmetic compositions according to the invention may be used, for example, as makeup products for the face and/or body, and/or nails.

The cosmetic compositions according to the invention may be used, for example, as care, cleansing and/or sun protection products for the face and/or body and/or nails with a liquid to semi-liquid consistency, such as milks, creams of varying smoothness, cream gels or pastes.

They may optionally be packaged in aerosol form and may be in the form of a mousse or a spray.

The compositions according to the invention in the form of vaporizable fluid lotions in accordance with the invention are applied to the skin or hair in the form of fine particles by means of pressurizing devices.

The devices suitable for use in the invention are well known to those skilled in the art and comprise non-aerosol pumps or “atomizers”, aerosol containers comprising a propellant and aerosol pumps using compressed air as propellant. These devices are described in patents U.S. Pat. No. 4,077,441 and U.S. Pat. No. 4,850,517.

The compositions packaged in aerosol form in accordance with the invention generally contain conventional propellants, for instance hydrofluoro compounds, dichlorodifluoromethane, difluoroethane, dimethyl ether, isobutane, n-butane, propane or trichlorofluoromethane. They are preferably present in quantities ranging from 15% to 50% by weight, relative to the total weight of the composition.

According to a specific embodiment of the invention, the composition according to the invention further comprises at least one deodorant active agent and/or at least one antiperspirant active agent.

\The term “deodorant active agent” refers to any substance that is capable of masking, absorbing, improving and/or reducing the unpleasant odor resulting from the decomposition of human sweat by bacteria.

The deodorant active agents may be bacteriostatic agents or bactericides that act on underarm odor microorganisms, such as 2,4,4′-trichloro-2′-hydroxydiphenyl ether (®Triclosan), 2,4-dichloro-2′-hydroxydiphenyl ether, 3′,4′,5′-trichlorosalicylanilide, 1-(3′,4′-dichlorophenyl)-3-(4′-chlorophenyl)urea (®Triclocarban) or 3,7,11-trimethyldodeca-2,5,10-trienol (®Farnesol); quaternary ammonium salts such as cetyltrimethylammonium salts, cetylpyridinium salts, polyols such as those of glycerol type, 1,3-propanediol (Zemea Propanediol® sold by DuPont Tate & Lyle Bio Products), 1,2-decanediol (Symclariol® from the company Symrise), glycerol derivatives, for instance caprylic/capric glycerides (Capmul MCM® from Abitec), glyceryl caprylate or caprate (Dermosoft GMCY® and Dermosoft GMC®, respectively from Straetmans), Polyglyceryl-2 caprate (Dermosoft DGMC® from Straetmans), biguanide derivatives, for instance polyhexamethylene biguanide salts; chlorhexidine and salts thereof; 4-phenyl-4,4-dimethyl-2-butanol (Symdeo MPP® from Symrise); cyclodextrins; chelating agents such as Tetrasodium Glutamate Diacetate (CAS #51981-21-6) sold under the trade name

Dissolvine GL-47-S® from AkzoNobel, EDTA (ethylenediaminetetraacetic acid) and DPTA (1,3-diaminopropanetetraacetic acid).

Among the deodorant active agents in accordance with the invention, mention may also be made of:
zinc salts, such as zinc salicylate, zinc phenolsulfonate, zinc pyrrolidonecarboxylate (more commonly known as zinc pidolate), zinc sulfate, zinc chloride, zinc lactate, zinc gluconate, zinc ricinoleate, zinc glycinate, zinc carbonate, zinc citrate, zinc chloride, zinc laurate, zinc oleate, zinc orthophosphate, zinc stearate, zinc tartrate, zinc acetate or mixtures thereof;
odor absorbers such as zeolites, especially silver-free metal zeolites, cyclodextrins, metal oxide silicates such as those described in patent application US 2005/063 928; metal oxide particles modified with a transition metal, as described in patent applications US 2005/084 464 and US 2005/084 474, aluminosilicates such as those described in patent application EP 1 658 863, chitosan-based particles such as those described in patent U.S. Pat. No. 6,916,465;
sodium bicarbonate;
salicylic acid and derivatives thereof such as 5-n-octanoylsalicylic acid;
alum;
triethyl citrate.
and mixtures thereof.

The deodorant active agents may preferably be present in the compositions according to the invention in weight proportions ranging from 0.01% to 10% by weight relative to the total weight of the composition.

The term “antiperspirant active agent” means a salt which, by itself, has the effect of reducing the flow of sweat, of reducing the sensation on the skin of moisture associated with human sweat or of masking human sweat.

Among the antiperspirant active agents, mention may be made of the antiperspirant salts or complexes of aluminum and/or of zirconium, preferably chosen from aluminum halohydrates; aluminum zirconium halohydrates, complexes of zirconium hydroxychloride and of aluminum hydroxychloride with or without an amino acid, such as those described in patent U.S. Pat. No. 3,792,068.

Among the aluminum salts, mention may in particular be made of aluminum chlorohydrate in activated or unactivated form, aluminum chlorohydrex, the aluminum chlorohydrex-polyethylene glycol complex, the aluminum chlorohydrex-propylene glycol complex, aluminum dichlorohydrate, the aluminum dichlorohydrex-polyethylene glycol complex, the aluminum dichlorohydrex-propylene glycol complex, aluminum sesquichlorohydrate, the aluminum sesquichlorohydrex-polyethylene glycol complex, the aluminum sesquichlorohydrex-propylene glycol complex, aluminum sulfate buffered with sodium aluminum lactate.

Among the aluminum zirconium salts, mention may be made in particular of aluminum zirconium octachlorohydrate, aluminum zirconium pentachlorohydrate, aluminum zirconium tetrachlorohydrate and aluminum zirconium trichlorohydrate.

The complexes of zirconium hydroxychloride and of aluminum hydroxychloride with an amino acid are generally known as ZAG (when the amino acid is glycine). Among these products, mention may be made of the aluminum zirconium octachlorohydrex-glycine complexes, the aluminum zirconium pentachlorohydrex-glycine complexes, the aluminum zirconium tetrachlorohydrex-glycine complexes and the aluminum zirconium trichlorohydrex-glycine complexes.

Aluminum sesquichlorohydrate is in particular sold under the trade name Reach 301® by the company Summitreheis.

Among the aluminum and zirconium complexes, mention may be made of the complexes of zirconium hydroxychloride and of aluminum hydroxychloride with an amino acid such as glycine, having the INCI name: Aluminium Zirconium Tetrachlorohydrex Gly, for example the product sold under the name Reach AZP-908-SUF® by the company Summitreheis.

Use will more particularly be made of the aluminum chlorohydrate sold under the trade names Locron S FLA®, Locron P and Locron L.ZA by the company Clariant; under the trade names Microdry Aluminum Chlorohydrate®, Micro-Dry 323®, Chlorhydrol 50, Reach 103 and Reach 501 by the company Summitreheis; under the trade name Westchlor 200® by the company Westwood; under the trade name Aloxicoll PF 40® by the company Guilini Chemie; Cluron 50%® by the company Industria Quimica Del Centro; or Clorohidroxido Aluminio SO A 50%® by the company Finquimica.

As other antiperspirant active agent, mention may be made of expanded perlite particles such as those obtained by the expansion process described in patent U.S. Pat. No. 5,002,698.

The perlites that may be used according to the invention are generally aluminosilicates of volcanic origin and have as the composition:

70.0-75.0% by weight of silica SiO₂

12.0-15.0% by weight of oxide of aluminum oxide Al₂O₃

3.0-5.0% of sodium oxide Na₂O

3.0-5.0% of potassium oxide K₂O

0.5-2% of iron oxide Fe₂O₃

0.2-0.7% of magnesium oxide MgO

0.5-1.5% of calcium oxide CaO

0.05-0.15% of titanium oxide TiO₂.

Preferably, the perlite particles used will be ground; in this case, they are known as Expanded Milled Perlite (EMP). They preferably have a particle size defined by a median diameter D50 ranging from 0.5 to 50 μm and preferably from 0.5 to 40 μm.

Preferably, the perlite particles used have a loose bulk density at 25° C. ranging from 10 to 400 kg/m³ (standard DIN 53468) and preferably from 10 to 300 kg/m³.

Preferably, the expanded perlite particles according to the invention have a water absorption capacity, measured at the wet point, ranging from 200% to 1500% and preferably from 250% to 800%.

The wet point corresponds to the amount of water which has to be added to 1 g of particle in order to obtain a homogeneous paste. This method derives directly from the oil uptake method applied to solvents. The measurements are taken in the same manner by means of the wet point and the flow point, which have, respectively, the following definition:

wet point: mass expressed in grams per 100 g of product corresponding to the production of a homogeneous paste during the addition of a solvent to a powder;

Flow point: mass expressed in grams per 100 g of product above which the amount of solvent is greater than the capacity of the powder to retain it. This is reflected by the production of a more or less homogeneous mixture which flows over the glass plate.

The wet point and the flow point are measured according to the following protocol:

Protocol for measuring the water absorption.

1) Equipment used

Glass plate (25×25 mm)

Spatula (wooden shaft and metal part, 15×2.7 mm)

Silk-bristled brush

Balance

2) Procedure

The glass plate is placed on the balance and 1 g of perlite particles is weighed out. The beaker containing the solvent and the liquid sampling pipette is placed on the balance. The solvent is gradually added to the powder, the whole being regularly blended (every 3 to 4 drops) with the spatula.

The weight of solvent needed to obtain the wet point is noted. Further solvent is added and the weight which makes it possible to reach the flow point is noted. The average of three tests will be determined.

The expanded perlite particles sold under the trade names Optimat 1430 OR or Optimat 2550 by the company World Minerals will be used in particular.

The antiperspirant active agents may be present in the composition according to the invention in a proportion of from 0.001% to 30% by weight and preferably in a proportion of from 0.5% to 25% by weight relative to the total weight of the composition.

Throughout the description, including the claims, the term “comprising a” should be understood as being synonymous with “comprising at least one”, unless otherwise specified.

The terms “between . . . and . . . ” and “ranging from . . . to . . . ” should be understood as being inclusive of the limits, unless otherwise specified.

In the description and the examples, the percentages are percentages by weight. The ingredients are mixed in the order and under the conditions that are easily determined by those skilled in the art.

### EXAMPLES

### Example 1

### Preparation of a Synthetic Phyllosilicate that is Suitable for use in the Invention

A synthetic phyllosilicate according to the invention is prepared according to the technology described in example 1 of patent application FR 2 977 580 from page 21, line 26 to page 23, line 20.

In the event when the phyllosilicate is in the form of an aqueous gel, the process was followed until the hydrogel formation without the drying step by lyophilization (from page 21 line 26 to page 22 line 29 of document FR 2 977 580).

Analysis of the x-ray diffractogram was performed with the aid of the materials and method used for the x-ray diffraction analyses that are detailed in patent application FR 2 977 580.

A characteristic diffraction line at 9.77 Å is observed.

The compositions according to the invention illustrated in the examples that follow comprise a synthetic phyllosilicate in accordance with the invention as obtained in this example 1.

### Example 2

### Compositions

2.1 Compositions According to the Invention

2.1.1 In compositions 1 to 5 according to the invention, different types of components have been used in the oil phase.

Emulsions 1 to 5 were prepared according to the following protocol:

1) In water, with stirring, the synthetic phyllosilicate gel having molecular formula Mg₃Si₄O₁₀(OH)₂ obtained according to example 1 and optionally an additional gellant are dispersed with stirring in a Rayneri;

2) The oil phase is mixed in the Rayneri;

3) The emulsion is formed at a temperature of 20-25° C., if there is no solid fatty substance at said temperature, with stirring in the Turax (9200 rpm, 20 min);

4) The preservative is added after cooling.

| Ingredients/INCI | Composition 1 | Composition 2 | Composition 3 | Composition 4 | Composition 5 |
|---|---|---|---|---|---|
| name | (invention) | (invention) | (invention) | (invention) | (invention) |
| Synthetic | 24.39 (2% | 24.39 (2% | 24.39 (2% | 24.39 (2% | 24.39 (2% |
| phyllosilicate gel | AI*) | AI*) | AI*) | AI*) | AI*) |
| PDMS 10 cst | 2.50 | 5.00 | — | — | — |
| (ELEMENT14 PDMS | | | | | |
| 10-A ®) | | | | | |
| Dicaprylyl ether | 2.50 | — | 5.00 | — | — |
| (CETIOL OE ®) | | | | | |
| Isohexadecane | — | — | — | 5.00 | — |
| (ISOHEXADECANE ®) | | | | | |
| Isopropyl palmitate | — | — | — | — | 5.00 |
| (LEXOL IPP NF ®) | | | | | |
| Demineralized water | qs 100 | qs 100 | qs 100 | qs 100 | qs 100 |
| Phenoxyethanol | 0.70 | 0.70 | 0.70 | 0.70 | 0.70 |
| (NEOLONE PH 100 ® - | | | | | |
| PRESERVATIVE) | | | | | |
| 24 hr stability | OK | OK | OK | OK | OK |
| at room temperature | | | | | |
| 20-25° C. | | | | | |
| After redispersion | Homogeneous | Homogeneous | Homogeneous | Homogeneous | Homogeneous |
| with a spatula | | | | | |

% AI* percentage by weight of active ingredient of synthetic phyllosilicate particles in water

Compositions 1 to 5 are stable and homogeneous.

2.1.2 Compositions 6, 7 and 8 According to the Invention

Compositions 6, 7 and 8 were prepared under the same conditions as previously in Example 2.1.1.

| | Composition 6 | Composition 7 | Composition 8 |
|---|---|---|---|
| Ingredients/INCI name | (invention) | (invention) | (invention) |
| Synthetic | 24.39 (2% AI*) | 24.39 (2% AI*) | 48.78 (4% AI*) |
| phyllosilicate gel | | | |
| PDMS 10 cst | 10.0 | 25.0 | 10.0 |
| (ELEMENT14 PDMS | | | |
| 10-A ®) | | | |
| Dicaprylyl ether | 10.0 | 25.0 | 10.0 |
| (CETIOL OE ®) | | | |
| Demineralized water | qs 100 | qs 100 | qs 100 |
| Phenoxyethanol | 0.7 | 0.7 | 0.7 |
| (NEOLONE PH | | | |
| 100 ® - | | | |
| PRESERVATIVE) | | | |
| 24 hr stability | OK | OK | OK |
| 20-25° C. | | | |
| 2 month stability | OK | OK | OK |
| 20-25° C. | | | |
| After redispersion with | Homogeneous | Homogeneous | Homogeneous |
| a spatula | Macro drop | Macro drop | |

% AI* percentage by weight of active ingredient of synthetic phyllosilicate particles in water

Compositions 6, 7 and 8 are stable and homogeneous.

2.1.3 In Composition 9 According to the Invention, Aluminum Chlorohydrate was used

Composition 9 was prepared under the same conditions as Example 2.1.1.

| | Composition 9 |
|---|---|
| Ingredients/INCI name | (invention) |
| Synthetic phyllosilicate gel | 24.39 (2% AI*) |
| PDMS 10 cst | 2.50 |
| (ELEMENT14 PDMS 10-A ®) | |
| Dicaprylyl ether | 2.50 |
| (CETIOL OE ®) | |
| Demineralized water | qs 100 |
| Phenoxyethanol | 0.7 |
| (NEOLONE PH 100 ® - | |
| PRESERVATIVE) | |
| Aluminum chlorohydrate | 30 |
| (CHLORHYDROL 50 ®) | |
| 24 hr stability at 20-25° C. | OK |
| Stability after 2 months at 20-25° C. | OK |
| After redispersion with a spatula | Homogeneous |

% AI* percentage by weight of active ingredient of synthetic phyllosilicate particles in water

Composition 9 is stable and homogeneous.

2.1.4 Compositions 10, 11 and 12 According to the Invention

Compositions 10, 11 and 12 were prepared under the same conditions as previously in Example 2.1.1.

| | Compo- | Compo- | Compo- |
|---|---|---|---|
| | sition 10 | sition 11 | sition 12 |
| Ingredients/INCI name | (invention) | (invention) | (invention) |
| Synthetic | 24.39 (2% AI*) | 24.39 (2% AI*) | 24.39 (2% AI*) |
| phyllosilicate gel | | | |
| PDMS 10 cst | 2.50 | 2.50 | 10.00 |
| (ELEMENT14 PDMS | | | |
| 10-A ®) | | | |
| Dicaprylyl ether | 2.50 | 2.50 | — |
| (CETIOL OE ®) | | | |
| Demineralized water | qs 100 | qs 100 | qs 100 |
| Phenoxyethanol | 0.70 | 0.70 | 0.70 |
| (NEOLONE PH | | | |
| 100 ®- | | | |
| PRESERVATIVE) | | | |
| HYDROXYPROPYL | — | 3.00 | — |
| STARCH | | | |
| PHOSPHATE | | | |
| STRUCTURE XL | | | |
| C18-38 Alkyl | — | — | 3.00 |
| Hydroxystearoyl | | | |
| Stearate | | | |
| (KESTER WAX K 82 | | | |
| P ®) | | | |
| 24 hr stability at | OK | OK | OK |
| 20-25° C. | | | |
| Stability after | OK | OK | OK |
| 2 months | | | |
| at 20-25° C. | | | |
| After redispersion | Homogeneous | Homogeneous | Homogeneous |
| with a spatula | | | |

% AI* percentage by weight of active ingredient of synthetic phyllosilicate particles in water

Compositions 10, 11 and 12 are stable and homogeneous.

2.1.5 In Composition 13 According to the Invention, a Synthetic Phyllosilicate Suitable for use in the Invention is used in Powder Form.

| | Composition 13 |
|---|---|
| Ingredients | Weight % |
| water | qs 100 |
| Liquid paraffin | 29.9 |
| Synthetic phyllosilicate (powder) | 30.1 |

Composition 13 is stable and homogeneous at 20-25° C. after two months.

2.2 Comparative Compositions 14 and 15

The following compositions 14 and 15 outside the invention are comparative compositions comprising respectively as powder natural talc or polymethylmethacrylate (PMMA).

| | Examples | |
|---|---|---|
| | Composition 14 | Composition 15 |
| | (comparative) | (comparative) |
| Natural talc (17 μm) | 2.0 | 2.0 |
| Polymethyl methacrylate | — | 2.0 |
| (SEPIMAT H 10 ®) | | |
| PDMS 10 cst | 2.5 | 2.5 |
| (ELEMENT14 PDMS 10- | | |
| A ®) | | |
| Dicaprylyl ether | 2.5 | 2.5 |
| (CETIOL OE ®) | | |
| Demineralized water | 62.3 | 62.3 |
| Phenoxyethanol | 0.7 | 0.7 |
| (NEOLONE PH 100 ® - | | |
| PRESERVATIVE) | | |
| Stability after 24 h and 20-25° C. | DESTABILIZED | DESTABILIZED |
| | FORMULA: | FORMULA: |
| | powder at the | powder at the |
| | bottom, milky | bottom, milky |
| | upper phase | upper phase |

Compositions 14 and 15 comprising natural talc or PMMA are destabilized after 24 hours at 20-25° C.

1. A Pickering emulsion comprising:
(a) at least one synthetic phyllosilicate having molecular formula Mg₃Si4O₁₀(OH)₂;
(b) at least one aqueous phase; and
(c) at least one oil phase.

2. The emulsion as claimed in claim 1, presenting an X-ray diffraction line greater than 9.4 Å and less than or equal to 9.8 Å.

3. The emulsion as claimed in claim 1, presenting an infrared absorption band at 7200 cm⁻¹, corresponding to the stretching vibration attributed to silanol groups Si—OH at the edge of the phyllosilicate sheets.

4. The emulsion as claimed in claim 1, said emulsion having no infrared absorption band at 7156 cm⁻¹.

5. The emulsion as claimed in claim 1, wherein the synthetic phyllosilicate is used in the form of an aqueous or aqueous-alcohol gel.

6. The emulsion as claimed in claim 5, wherein the synthetic phyllosilicate is present in a quantity ranging from 0.5% to 10% by weight of active ingredient relative to the total weight of the composition for 0.3% to 50% by weight of oil phase.

7. The emulsion as claimed in claim 1, wherein the synthetic phyllosilicate is used in the form of a powder.

8. The emulsion as claimed in claim 7, wherein the phyllosilicate is present in a quantity ranging from 0.1% to 40% by weight of active ingredient relative to the total weight of the composition.

9. The emulsion as claimed in claim 1, wherein said emulsion is in the form of an oil-in-water or water-in-oil emulsion.

10. The emulsion as claimed in claim 1, wherein said emulsion further contains at least one water-soluble polysaccharide.

11. The emulsion as claimed in claim 1, wherein said emulsion also contains at least one deodorant active and/or at least one antiperspirant active.

12. A method for stabilizing a Pickering emulsion comprising:
(a) at least one synthetic phyllosilicate having molecular formula Mg₃Si₄O₁₀(OH)₂;
(b) at least one aqueous phase; and
(c) at least one oil phase,
using a synthetic phyllosilicate having molecular formula Mg₃Si₄O₁₀(OH)₂.

13. A process for preparing a Pickering emulsion comprising:
(a) at least one synthetic phyllosilicate having molecular formula Mg₃Si₄O₁₀(OH)₂;
(b) at least one aqueous phase; and
(c) at least one oil phase,
the process comprising at least the following steps:
1) In water, with stirring, a synthetic phyllosilicate having molecular formula Mg₃Si₄O₁₀(OH)₂ is dispersed with stirring in a Rayneri;
2) The aqueous phase is mixed with the oil phase;
3) The emulsion is formed;
(i) at a temperature of 20-25° C., if there is no fatty substance that is solid at said temperature, with stirring;
(ii) at a higher temperature than the melting point of said solid fatty substance.

14. A cosmetic treatment process comprising the application on skin and/or nails of a Pickering emulsion comprising:
(a) at least one synthetic phyllosilicate having molecular formula Mg₃Si₄O₁₀(OH)₂;
(b) at least one aqueous phase; and
(c) at least one oil phase.
